(19) United States
(12) Patent Application Publication
(10) Pub. No.: US 2015/0265169 A1
(43) Pub. Date: Sep. 24, 2015

(54) END CAPS FOR BLOOD PRESSURE CUFFS AND BLOOD PRESSURE CUFFS THEREWITH

(71) Applicant: SHENZHEN MINDRAY BIO-MEDICAL ELECTRONICS CO., LTD., Shenzhen (CN)

(72) Inventor: Jiao Kun, Shenzhen (CN)

(73) Assignee: SHENZHEN MINDRAY BIO-MEDICAL ELECTRONICS CO., LTD.

(21) Appl. No.: 14/733,755

(22) Filed: Jun. 8, 2015

Related U.S. Application Data
(63) Continuation of application No. PCT/CN2013/085639, filed on Oct. 22, 2013.

(30) Foreign Application Priority Data
Dec. 6, 2012 (CN) 201210520093.1

(51) Int. Cl. A61B 5/022

(52) U.S. Cl. CPC A61B 5/02233

(57) ABSTRACT

This disclosure relates generally to medical devices. An end cap for a blood pressure cuff can comprise a flexible outer covering part, wherein an inner hole may be arranged on said outer covering part, said inner hole may be a blind hole, and said outer covering part is capable of clamping an outer wall of an air plug arranged on the blood pressure cuff by interference fit between said inner hole and said air plug; the end cap may be inserted into an inner wall and/or clamp an outer wall of the air plug to seal the open end of the air plug, thus small particles, such as dust, and cleaning and disinfectant liquid cannot enter into the blood pressure cuff and thus cause an air path jam and corrode air path components during deflating, thereby prolonging the life of the blood pressure cuff.

### TECHNICAL FIELD

This disclosure relates generally to medical devices, particularly to external end caps for blood pressure cuffs and blood pressure cuffs therewith.

### SUMMARY

Disclosed herein are embodiments of end caps for blood pressure cuffs and blood pressure cuffs with them.

In one aspect, an end cap for a blood pressure cuff is provided, which may include a flexible outer covering part. An inner hole may be arranged on said outer covering part. The inner hole may be a blind hole, and said outer covering part is capable of clamping an outer wall of an air plug arranged on the blood pressure cuff by an interference fit between said inner hole and said air plug.

In one embodiment, the end cap may further comprise an inner plug which may be arranged at the bottom of said inner hole.

In one embodiment, a radial dimension of said inner plug may gradually increase from one end near an open end of said inner hole to the other end, and said inner plug may be inserted into said air plug by interference fit after said air plug is inserted into said end cap.

In one embodiment, the end face of said inner plug near the open end of said inner hole may be connected to a side of said inner plug by a rounded transition zone.

In one embodiment, said inner plug may be shorter than said outer covering part.

In one embodiment, said outer covering part may radially extend from the base end of said inner hole to form a guiding part, and said guiding part may protrude from an outer edge of said outer covering part.

In one embodiment, the end cap may further comprise a mounting part, where one end of said mounting part may be connected to said outer covering part and the other end may be connected to said air plug.

In one embodiment, said mounting part may include a mounting loop and a strip. One end of said strip may be connected to said outer covering part and the other end may be connected to said mounting loop. Said mounting loop may be located outside said air plug.

In one embodiment, said end cap may be made of soft polymeric material.

In one embodiment, the thickness of said outer covering part may increase from the open end of said inner hole to the other end.

In another aspect, a blood pressure cuff may comprise an inner air guiding tube, an air plug and the end cap as described above, where one end of said air plug may be inserted into said inner air guiding tube and the other end may be sealed by said end cap.

In one embodiment, a raised edge may be arranged around the middle of said air plug, and said end cap may further comprise a mounting loop located between said inner air guiding tube and said raised edge.

In one embodiment, an annular groove may be arranged around the middle of the outer wall of said air plug.

In one embodiment, said mounting loop may be directly installed on said inner air guiding tube or said air plug.

In one embodiment, said mounting loop may be integrally formed on said inner air guiding tube or said air plug.

The end cap and the blood pressure cuff described above may clamp the outer wall of the air plug arranged on the blood pressure cuff by interference fit between the inner hole and the air plug, therefore the outer covering part may cover the outer wall of the air plug firmly. Thus, small particles, such as dust, cannot enter into the blood pressure cuff through the air plug and cause an air path jam during deflating, and cleaning and disinfectant liquid cannot enter into the blood pressure cuff during cleaning and disinfecting and thereby corrode the air path components during deflating, thereby prolonging the life of the blood pressure cuff.

The thickness of the outer covering part may increase from the open end of the inner hole to the other end and the side wall of the outer covering part may be the thickest at the base end of the inner hole, thus providing enough holding force after the air plug is inserted into the inner hole so as to prevent the end cap from detaching from the air plug easily. The structure of the outer covering part that the side wall is thinner at the open end and thicker at the base end improves convenience and reliability of using the end cap.

### BRIEF DESCRIPTION OF THE DRAWINGS

FIG. 1 shows a schematic diagram of an end cap for a blood pressure cuff according to an embodiment;

FIG. 2 is a partial view of the internal structure of the end cap according to an embodiment;

FIG. 3 shows a schematic diagram of the end cap installed on a blood pressure cuff according to an embodiment;

FIG. 4 shows an assembly drawing of the end cap installed on the blood pressure cuff according to an embodiment;

FIG. 5 shows a schematic diagram of the end cap installed on the blood pressure cuff when the blood pressure cuff is in use according to an embodiment; and

FIG. 6 is an internal view of an air plug sealed by the end cap according to an embodiment.

### DETAILED DESCRIPTION

A blood pressure cuff is a key component of a non-invasive blood pressure cuff. The blood pressure cuff, which directly contacts a human body and collects blood pressure fluctuation signals, is a sensitive element for blood pressure measurement. The blood pressure cuff may comprise an inflating portion, an air duct and an air plug. The inflating portion may be inflated by air and detect human arterial pulses during blood pressure measurement. The inflating portion may be connected to the air plug by the air duct. The air plug may be a connection port for connecting the blood pressure cuff and an external air duct.

For a conventional blood pressure cuff, small particles, such as dust, will enter into the cuff through the air plug and cause an air path jam during deflating, and cleaning and disinfectant liquid will enter into the cuff during cleaning and disinfecting and may corrode air path components during deflating, thereby shortening the life of the non-invasive blood pressure cuff.

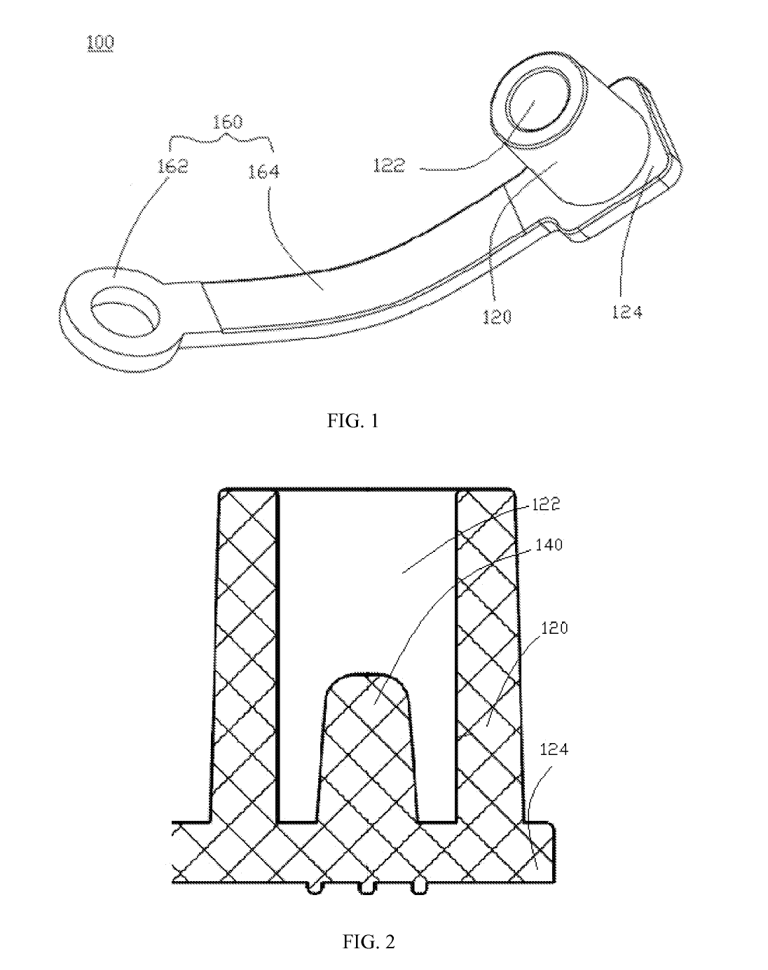

As shown in FIGS. 1 and 2, in one embodiment, an end cap 100 for a blood pressure cuff may include an outer covering part 120, an inner plug 140 and a mounting part 160.

As shown in FIGS. 1, 2 and 6, an inner hole 122 may be arranged in the outer covering part 120, where the inner hole 122 may be a blind hole. The diameters of the blind hole in the cross sections thereof may be the same or change gradually. In other embodiments, the diameters of the blind hole may change in other ways. The thickness of the outer covering part 120 may increase from the open end of the inner hole 122 to the other end. The outer covering part 120 may clamp an outer side of an air plug 200 of a blood pressure cuff by interference fit between the outer covering part 120 and the inner hole 122.

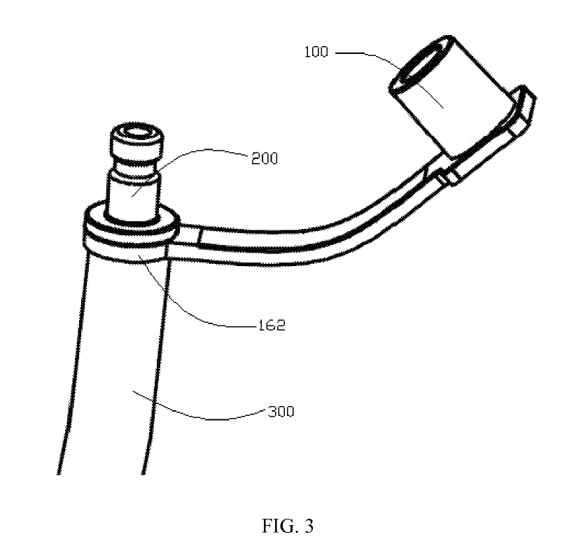

As shown in FIGS. 3, 5 and 6, the end cap 100 may cover the air plug 200 of the blood pressure cuff by the inner hole 122. When the blood pressure cuff is in use, the air plug 200 may be inserted into the inner hole 122. The outer covering part 120 may be elastic, and the diameter of the inner hole 122 may be smaller than the radial dimension of the outer side of the air plug 200. The outer covering part 120 may be deformed elastically under radial pressure applied by the air plug 200 during inserting, the outer covering part 120 may cover the outer edge of the air plug 200 after the air plug 200 is inserted into the inner hole 122, and due to an elastic reset force generated by the outer covering part 120, the end cap 100 will not detach from the air plug 200 easily. Thus, cleaning and disinfectant liquid or small particles cannot enter into the air plug 200, the air path jam and corrosion of the air path components can be avoided, and the life of the blood pressure cuff can be prolonged.

In one embodiment, the thickness of the side wall of the outer covering part 120 may increase from the open end of the inner hole 122 to the other end. That is, the side wall of the outer covering part 120 may be thinner at the open end of the inner hole 122, so the outer covering part 120 may be easy to deform and the air plug 200 can be inserted into the inner hole 122 easily. The side wall of the outer covering part 120 may be thicker at the base end of the inner hole 122, thus it could provide enough holding force after the air plug 200 is inserted into the inner hole 122 so as to prevent the end cap 100 from detaching from the air plug 200 easily. Because the side wall of the outer covering part 120 is thinner at the open end and thicker at the base end the convenience and reliability of using the end cap 100 are improved.

As shown in FIG. 3, in one embodiment, the outer covering part 120 may have a cylindrical shape, which may provide balanced pressure for assembling the end cap 100 on the air plug 200. Of course, in other embodiments, the outer covering part 120 can have other shapes, such as a multilateral cylindrical shape, which will not affect the covering of the end cap 100 on the air plug 200.

As shown in FIG. 1, in one embodiment, the outer covering part 120 may extend from the base end of the inner hole 122 to form a guiding part 124, and the guiding part 124 may protrude from the outer edge of the outer covering part 120. An external force, applied on the guiding part 124 but not on the outer covering part 120 directly, may be used to seal the air plug 200 through the end cap 100 or to detach the air plug 200 from the end cap 100, thus the risk of destroying the outer covering part 120 can be avoided. In addition, it is easier for a user to apply the external force, such as by a finger, to the guiding part 124, especially when detaching the air plug 200 from the end cap 100. It can be understood by a person skilled in the art that the end cap 100 can seal the air plug 200 without the guiding part 124, which can also avoid cleaning and disinfectant liquid or small particles, such as dust, from entering into the air plug 200. So the guiding part 124 is not required.

As shown in FIG. 2, in one embodiment, the inner plug 140 may be arranged in the bottom of the inner hole 122. Thus, while the air plug 200 is being inserted into the inner hole 122, the inner plug 140 is inserted into the air plug 200. After being inserted, the outer edge of the air plug 200 may be sealed by the outer covering part 120 and the stoma of the air plug 200 may be clogged by the inner plug 140, thus a better waterproof and dustproof effect can be achieved.

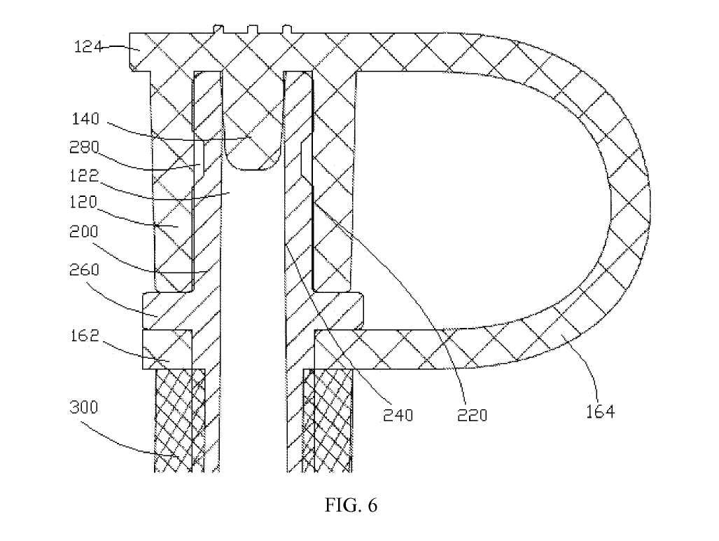

As shown in FIGS. 2 and 6, in one embodiment, the inner plug 140 may have a frustoconical shape. The radial dimension of the inner plug 140 may gradually increase from the end near the open end of the inner hole 122 to the other end. The radial dimension of the inner plug 140 at the end near the open end of the inner hole 122 may be smaller than a diameter of an inner wall 240 of the air plug 200, and the radial dimension of the inner plug 140 at the other end may be larger than the diameter of the inner wall 240 of the air plug 200. The fit type between the inner plug 140 and the stoma of the air plug 200 may gradually change from a clearance fit to an interference fit. Thus, the inner plug 140 can be inserted into the air plug 200 easily, and after the inner plug 140 is inserted securely into the air plug 200, the interference fit between the inner plug 140 and the inner wall 240 of the air plug 200 can improve the sealing effect and thereby further prevent the cleaning and disinfectant liquid or dust from entering into the air plug 200 and prolong the life of the blood pressure cuff.

As shown in FIGS. 2 and 6, in one embodiment, the end face of the inner plug 140 near the open end of the inner hole 122 may be connected to the side thereof by a rounded transition zone. Thus, it is easier for the inner plug 140 to be inserted into the air plug 200. Besides, it is not easy for the inner wall 240 of the air plug 200 to be damaged by the rounded transition zone when the inner plug 140 is inserted into the air plug 200, and the life of the blood pressure cuff can be prolonged. Of course, it is also possible that there is no such rounded transition zone.

As shown in FIGS. 2 and 6, in one embodiment, the inner plug 140 is shorter than the outer covering part 120. Of course, the inner plug 140 can be as long as or a little longer than the outer covering part 120. A raised edge 260 may be arranged around the middle of the air plug 200. When the blood pressure cuff is connected with an external air guiding tube 300, one end of the air plug 200 may be inserted into an inner air guiding tube 300 and the other end may be inserted into the external air guiding tube, and the raised edge 260 may be clamped between the inner air guiding tube 300 and the external air guiding tube 300. After the blood pressure cuff is detached from the external air guiding tube 300, the open end of the air plug 200 can be sealed by the end cap 100. The outer covering part 120 may clamp an outer wall 220 of the air plug 200 and the open end of the outer covering part 120 may fit the raised edge 260 closely, thereby the effect of waterproofing and dustproofing may be further improved and the blood pressure cuff can be placed easily. In the meantime, the inner plug 140 may be inserted into the air plug 200 with an interference fit, and the inner plug 140 is shorter than the outer covering part 120, so it is easier for the inner plug 140 to be inserted into the air plug 200 and material can be saved.

As shown in FIGS. 1 and 3, one end of the mounting part 160 may be connected to the outer covering part 120 and the other end may be connected to the air plug 200. When the end cap 100 is not needed by the blood pressure cuff, it can be taken off the air plug 200 without putting the end cap 100 into specialized equipment used for holding it; when the end cap 100 is needed by the blood pressure cuff, it can be easily found. It can be understood by a person skilled in the art that the mounting part 160 may be not necessary, which will not affect the use of the end cap 100.

As shown in FIGS. 3 to 5, in one embodiment, the mounting part 160 may include a mounting loop 162 and a strip 164. One end of the strip 164 may be connected to the outer covering part 120 and the other end of the strip 164 may be connected to the mounting loop 162. The mounting loop 162 may be located outside the air plug 200 and under the raised edge 260. After the air plug 200 is inserted into the inner air guiding tube 300, the mounting loop 162 may be between the inner air guiding tube 300 and the raised edge 260 and fit closely with the end face of the inner air guiding tube 300. Thus, the mounting loop 162 will not slide off the air plug 200 and it is easy for the user to take the mounting loop 162 off the air plug 200. When the air plug 200 is sealed by the end cap 100, the upper surface of the raised edge 260 may cover the outer covering part 120, which improves the sealing effect. In addition, the mounting loop 162 of the end cap 100 can be directly installed on the inner air guiding tube 300 or the air plug 200, or the end cap 100 can be integrally formed on the inner air guiding tube 300 or the air plug 200 without the mounting loop 162.

In other embodiments, the mounting part 160 can be connected to the outer covering part 120 and the air plug 200 by a chain or other means, or the mounting loop 162 can be located outside the inner air guiding tube 300.

It can be understood by a person skilled in the art that the mounting part 160 and the outer covering part 120 can be separate units. When used, the mounting part 160 and the outer covering part 120 can be connected by a snap or other detachable connection.

In some embodiments, the outer covering part 120 and the inner plug 140 are formed integrally, such as by way of injection molding. The arrangement that the outer covering part 120 and the inner plug 140 are formed integrally can improve the sealability and usability of the blood pressure cuff, simplify the manufacturing process and save costs. In other embodiments, the outer covering part 120 and the inner plug 140 can be connected through other means, such as an adhesive.

In some embodiments, the end cap 100 can be made of a soft polymeric material and formed by an injection molding process, etc. The end cap 100 made of soft polymeric materials may have organic and inorganic features, and other good performance characteristics, such as temperature resistance and weatherability and being physiologically inert. The end cap 100 will not adversely affect the air plug 200, and the life of the blood pressure cuff will be prolonged.

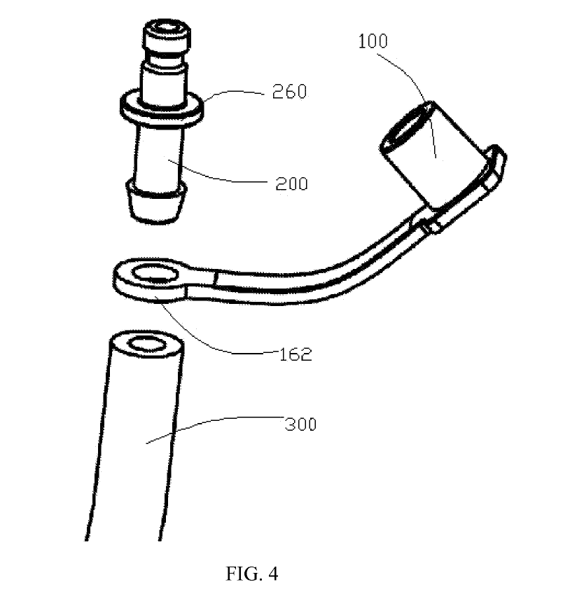

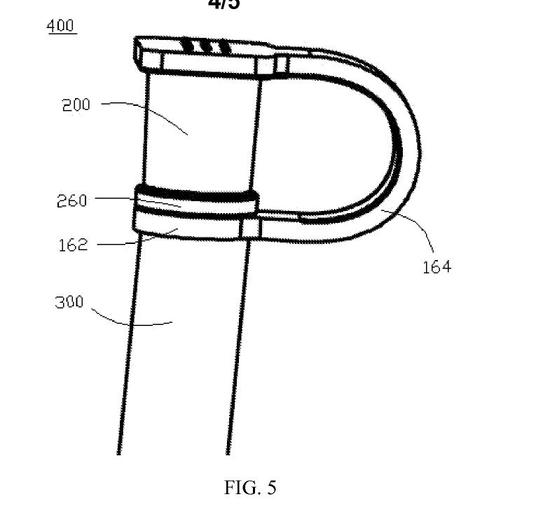

As shown in FIGS. 4 and 5, a blood pressure cuff 400 may include the inner air guiding tube 300, the air plug 200 and the end cap 100 described above. One end of the air plug 200 may be inserted into the inner air guiding tube 300 and the other end may be sealed by the end cap 100. The air plug 200 may be detachably sealed by the end cap 100. The air plug 200 may include the inner wall 240 and the outer wall 220; the end cap 100 can be inserted in the inner wall 240 and/or clamp the outer wall 220 to seal the open end of the air plug 200. Thus, small particles, such as dust, cannot enter into the blood pressure cuff 400 through the air plug 200 and thus cause an air path jam during deflating, and cleaning and disinfectant liquid cannot enter into the blood pressure cuff 400 during cleaning and disinfecting and thus corrode the air path components during deflating, thereby prolonging the life of the blood pressure cuff.

As shown in FIGS. 4 and 6, in one embodiment, an annular groove 280 may be arranged at the middle of outer wall 220. The outer covering part 120 may be a flexible member, so the outer covering part 120 may be deformed by being stretched to generate a reset force after the air plug 200 is inserted into the inner hole 122 of the outer covering part 120. After being inserted, the part of the outer covering part 120 corresponding to the annular groove 280 may contract due to the reset force, thus on the one hand a better sealing effect can be achieved, and on the other the air plug 200 can be sealed stably by the end cap 100 due to increased friction between the air plug 200 and the inner wall 240 of the outer covering part 120. Of course, it could be understood by a person skilled in the art that the air plug 200 can be sealed by the outer covering part 120 without the annular groove 280, so the annular groove 280 may be unnecessary.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, advantages and solutions to problems have been described above with regard to various embodiments and are not to be construed as a critical, required or essential feature or element. The scope of the present disclosure should, therefore, be determined by the following claims.

What is claimed is:

1. An end cap for a blood pressure cuff, comprising a flexible outer covering part, wherein an inner hole is arranged in said outer covering part, said inner hole is a blind hole, and said outer covering part is capable of clamping an outer wall of an air plug arranged on the blood pressure cuff by interference fit between said inner hole and said air plug.

2. The end cap of claim 1, further comprising an inner plug, wherein said inner plug is arranged at the bottom of said inner hole.

3. The end cap of claim 2, wherein a radial dimension of said inner plug gradually increases from an end near an open end of said inner hole to the other end, and said inner plug is insertable into said air plug by interference fit after said air plug is inserted into said end cap.

4. The end cap of claim 2, wherein an end face of said inner plug near an open end of said inner hole is connected to a side of said inner plug by a rounded transition zone.

5. The end cap of claim 2, wherein said inner plug is shorter than said outer covering part.

6. The end cap of claim 1, wherein said outer covering part extends from a base end of said inner hole to form a guiding part, and said guiding part protrudes from an outer edge of said outer covering part.

7. The end cap of claim 1, further comprising a mounting part, wherein one end of said mounting part is connected to said outer covering part and the other end is connected to said air plug.

8. The end cap of claim 7, wherein said mounting part includes a mounting loop and a strip, one end of said strip is connected to said outer covering part and the other end is connected to said mounting loop, and said mounting loop is located outside said air plug.

9. The end cap of claim 1, wherein said end cap is made of a soft polymeric material.

10. The end cap of claim 1, wherein a thickness of said outer covering part increases from an open end of said inner hole to the other end.

11. A blood pressure cuff, comprising an inner air guiding tube, an air plug and the end cap of claim 1, wherein one end of said air plug is inserted into said inner air guiding tube and the other end is sealed by said end cap.

12. The blood pressure cuff of claim 11, wherein a raised edge is arranged around the middle of said air plug, and said end cap further comprises a mounting loop located between said inner air guiding tube and said raised edge; or wherein an annular groove is arranged around the middle of an outer wall of said air plug.

13. The blood pressure cuff of claim 12, wherein said mounting loop is directly installed on said inner air guiding tube or said air plug.

14. The blood pressure cuff of claim 8, wherein said mounting loop is integrally formed on said inner air guiding tube or said air plug.
